(19) United States
(12) Patent Application Publication
(10) Pub. No.: US 2024/0041971 A1
(43) Pub. Date: Feb. 8, 2024

(54) A PROCESS OF PREPARATION OF FLOUR MIX AND ITS USE FOR THE TREATMENT OF DIABETES

(71) Applicant: DR DOZO LABORATORIES, Mohali, Punjab (IN)

(72) Inventor: Gurdial Singh ARORA, Mohali (IN)

(21) Appl. No.: 18/269,216

(22) PCT Filed: Dec. 22, 2021
(86) PCT No.: PCT/IN2021/051193

§ 371 Date: Jun. 22, 2023

(30) Foreign Application Priority Data
Dec. 23, 2020 (IN) 202011055968

(51) Int. Cl. A61K 36/899; A61K 36/48; A61P 3/10; A61P 17/02; A61K 9/06

(52) U.S. Cl. CPC A61K 36/899; A61K 36/48; A61P 3/10; A61P 17/02; A61K 9/06

(57) ABSTRACT

The present invention relates to a process for preparing a pharmaceutical preparation which is to be used for diabetes and diabetic complications treatment. The present invention also provides a method of treating diabetes and diabetic complications using Semolina. Triticum and Chickpea or the pharmaceutical preparation comprising them and the mixtures thereof. The present process is simple. economical. bio-friendly, and industrially applicable.

### FIELD THE INVENTION

The present invention relates to the field of pharmaceutical sciences. Accordingly, the present invention relates to a process for preparing a pharmaceutical preparation which is to be used for diabetes and diabetic complications treatment. The present invention also provides a method of treating diabetes and diabetic complications using Semolina, triticum and chickpea or the pharmaceutical preparation comprising them and the mixtures thereof. The present process is simple, economical, bio-friendly, and industrially applicable.

### BACKGROUND OF THE INVENTION

Diabetes mellitus is a chronic metabolic disorder of multiple aetiology, characterized by chronic hyperglycemia with disturbance of carbohydrate, fat and protein metabolism resulting from defects in insulin secretion, insulin action, or both. The effect of diabetes mellitus includes long-term damage, dysfunction, and failure of various organs. Diabetes mellitus is usually divided into two major categories:
Type 1 diabetes, (formerly insulin-dependent diabetes mellitus) encompasses the majority of cases which are primarily due to pancreatic islet beta-cell destruction, usually develop in childhood or adolescence and are prone to ketosis and acidosis. Type 1 diabetes accounts for around 10% of all diabetes.
Type 2 diabetes (formerly non-insulin-dependent diabetes mellitus) includes the common major form of diabetes which results from defect(s) in insulin secretion, almost always with a major contribution from insulin resistance. Type 2 diabetes accounts for around 90% of all diabetes.

A chronic condition that affects the way the body processes blood sugar (glucose). With type 2 diabetes, the body either does not produce enough insulin, or it resists insulin. Type 2 diabetes is a lifelong disease that keeps your body from using insulin the way it should. People with type 2 diabetes are said to have insulin resistance.

People who are middle-aged or older are most likely to get this kind of diabetes. It used to be called adult-onset diabetes. But type 2 diabetes also affects kids and teens, mainly because of childhood obesity. Type 2 is the most common type of diabetes. There are about 29 million people in the U.S. with type 2. Another 84 million have prediabetes, meaning their blood sugar (or blood glucose) is high but not high enough to be diabetes yet.

The possible diabetes complications include Cardiovascular disease, Nerve damage, Kidney damage, Eye damage, Foot damage, Skin conditions, Hearing impairment, Alzheimer's disease, depression, and other related disorders.

It is a brainchild of the inventor whose interest is in studying and converting edible material into active targeted therapies and taking care of critical and chronic diseases. That is how the inventor came across Chickpea, Triticum and Semolina and started studying for its Anti Arthritic properties. After performing further research and pre-clinical studies, the inventor observed its anti-arthritis activities that are too significant. Further, it was tested into humans which gives encouraging results in areas where present day therapies are not working. It is unique because it is derived from mixture of Semolina, Triticum and Chickpea and then the biotech process is involved bringing efficacy to the final product.

Chickpea, Semolina and Triticum are very commonly cultivated in various parts of the world. It can be grown in summer as well winter seasons. The fields they are grown in are close to the inventor's premises. The grains were first grinded and pulvaried to powder form and then biotechnologically processed. First use of this mixture was done in March 2000 to study its anti-arthritis properties. Raw material was tested for pesticides content and no contents of the pesticides were found.

The inventor in the present invention have developed a process for preparing the pharmaceutical preparation. The said preparation has shown results in the diabetes and diabetic complications treatment. Accordingly, the present invention provides a method of preparing the pharmaceutical preparation that can be utilized in the treatment of diabetes and diabetic complications. Also provided is the method of treating diabetes and diabetic complications using Semolina, Triticum and Chickpea or the pharmaceutical preparation comprising them and the mixtures thereof.

### OBJECTS OF THE PRESENT INVENTION

The main object of the present invention is to provide a process of preparing a pharmaceutical preparation comprising Semolina, triticum and chickpea and the combinations thereof. Another object of the present invention is to provide a process of preparing a pharmaceutical preparation which have applications in the treatment of diabetes and related complications of diabetes.

Another object of the present invention is to provide a method of treating diabetes and related complications of diabetes using Semolina, triticum and chickpea or the pharmaceutical preparation comprising them.

Another object of the present invention is to provide a process which is simple, economical, bio-friendly, and industrially applicable.

### SUMMARY OF THE INVENTION

The present invention relates to a process for preparing a pharmaceutical preparation which is to be used for diabetes and diabetic complications treatment. The present invention also provides a method of treating diabetes and diabetic complications using Semolina, Triticum and Chickpea or the pharmaceutical preparation. comprising them and the mixtures thereof. The present process is simple, economical, bio-friendly, and industrially applicable.

### DETAILED DESCRIPTION OF THE INVENTION

It is the novel idea by the inventor and used for said diseases. Accordingly, the present invention relates to a process for preparing a pharmaceutical preparation which is to be used in the treatment of Diabetes and diabetic complications. The process comprises of the steps of mixing Triticum, Chickpea and Semolina (obtained by grinding grains) with water and vigorously stirred. The solution is then kept at a temperature 27 ±3° C. and is kept at the temperature for 16 hours, then subjected to further stirring and put into dryers at the temperature 80° C. for 48 hours. The material so obtained is further grinded to obtain very fine powder.

Furthermore, the solution was dried slowly at less than 60 degrees for 48 hours. It demonstrated very good Anti diabetic activity in studies.

According to a preferred embodiment of the present invention, the flours are mixed with distilled water, more preferably double distilled water.

According to another preferred embodiment of the invention, the solution flour is being kept at 27±3° C. for 16±2 hours, subjected to drying at the temperature of 80° C. for 48 hours.

According to another embodiment of the present invention, the solution was dried slowly at less than 60 degrees for 48 hours.

According to another preferred embodiment of the present invention, Triticum, Semolina and chickpea flour and water are mixed in 1:2 ratio respectively. Various means were tried to obtain solids after 2^{nd} step with drying by known methods.

Another embodiment of the present invention provides a method of treating diabetes and diabetic complications using Semolina, Triticum and Chickpea or the pharmaceutical preparation comprising them and the mixtures thereof.

Another embodiment of the present invention provides a use of Semolina, Triticum and Chickpea or the pharmaceutical preparation comprising them and the mixtures thereof in the treatment of diabetes and diabetic complications.

Another embodiment of the present invention provides a method of treating diabetes and diabetic complications using the pharmaceutical preparation which comprises Semolina, Triticum and Chickpea or the mixtures thereof.

Another embodiment of the present invention provides a process of preparing the pharmaceutical preparation wherein the process is simple, economical, bio-friendly, and industrially applicable.

A preferred embodiment of the present invention provides a pharmaceutical preparation of Semolina, Triticum and Chickpea or the mixtures thereof which is be used in the treatment of diabetes and diabetic complications.

The terms “treatment,” “treat,” “treating,” and the like, are meant to include slowing or reversing the progression of a disease or disorder. These terms also include alleviating, ameliorating, attenuating, eliminating, or reducing one or more symptoms of a disease or disorder or condition, even if the disease or disorder or condition is not actually eliminated and even if progression of the disease or disorder or condition is not itself slowed or reversed.

As explained above, the pharmaceutical preparation of the invention is useful in treating or preventing diabetes and related complications of diabetes. The present invention therefore provides pharmaceutical preparation for use in treatment of diabetes and related complications of diabetes. Also provided is a method for treating a patient suffering from diabetes or related complications, which method comprises administering to said patient an effective amount of a pharmaceutical preparation. Further provided is the use of a pharmaceutical preparation, in the manufacture of a medicament for use in treatment of diabetes and related complications of diabetes.

The main embodiment of the present invention is to provide a process of preparing a pharmaceutical preparation, wherein the process comprising the steps of:
a) Mixing flours of Triticum, Chickpea and Semolina with water and stirred vigorously to form a solution.
b) Keeping the solution of step (a) at an appropriate temperature range of 25 to 35° C. for adequate time ranging 12 to 19 hours to form another solution.
c) The solution of step (b) is subjected to vigorous stirring, following by drying at temperature range of 70 to 90° C. for the time range of 40 to 60 hours to obtain a mixture.
d) The mixture of step (c) is further grinded to obtain very fine powder.
e) The powder of step (d) is dried slowly at temperature range of 0 to 65° C. for the time range of 42 to 55 hours to obtain a pharmaceutical preparation.

In another embodiment of the present invention, flours of Triticum, Chickpea and Semolina are obtained by grinding the grains.

In another embodiment of the present invention, the water is distilled water.

In another embodiment of the present invention, the distilled water is more preferably double distilled water.

In another embodiment of the present invention, Triticum, Semolina and chickpea flour and water are mixed in a ratio of 1:1 to 1:4, preferably, 1:2.

In another embodiment of the present invention, the appropriate temperature of step (b) is in the range of 26 to 32° C.

In another embodiment of the present invention, the appropriate temperature of step (b) is preferably 27±3° C.

In another embodiment of the present invention, the adequate time of step (b) ranges from 15 to 18 hours.

In another embodiment of the present invention, the adequate time of step (b) is 16 hours.

In another embodiment of the present invention, the drying of step (c) is carried out at temperature range of 75 to 85° C., preferably 78 to 80° C.

In another embodiment of the present invention, the temperature of step (c) is 80° C.

In another embodiment of the present invention, the time range of step (c) is 45 to 55 hours, more preferably, 48 hours.

In another embodiment of the present invention, the temperature of step (e) is less than 62° C., more preferably less than 60° C.

In another embodiment of the present invention, the time range of step (e) is 46 to 52 hours, more preferably, 48 hours.

In another embodiment of the present invention, the pharmaceutical preparation has application in the treatment of diabetes and diabetic complications.

Another embodiment of the present invention provides a method of treating diabetes and diabetic complications using Semolina, triticum and chickpea or the pharmaceutical preparation comprising same.

Another embodiment of the present invention provides use of the pharmaceutical preparation as prepared in the process for the treatment of diabetes and diabetic complications.

Yet another embodiment of the present invention provides a process of preparing a pharmaceutical preparation, wherein the process comprising the steps of:
a. Mixing flours of Triticum, Chickpea and Semolina with water and stirred vigorously to form a solution.
b. Keeping the solution of step (a) at an appropriate temperature of 27±3 ° C. for 16 hours to form another solution.
c. The solution of step (b) is subjected to vigorous stirring, following by drying at temperature of 80° C. for 48 hours to obtain a mixture.
d. The mixture of step (c) is further grinded to obtain very fine powder.
e. The powder of step (d) is dried slowly at temperature of less than 60° C. for 48 hours to obtain a pharmaceutical preparation.

In another embodiment of the present invention, the pharmaceutical preparation has application in the treatment of diabetes and diabetic complications.

Another embodiment of the present invention provides a method of treating diabetes and diabetic complications using Semolina, triticum and chickpea or the pharmaceutical. preparation comprising the same.

Another embodiment of the present invention provides the use of the pharmaceutical preparation as prepared in process for the treatment of diabetes and diabetic complications.

Yet another embodiment of the present invention provides a process of preparing a pharmaceutical preparation, wherein the process comprising the steps of:
a. Mixing flours of Triticum, Chickpea and Semolina with water and stirred vigorously to form a solution.
b. Keeping the solution of step (a) at an appropriate temperature of 27±3 ° C. for 16 hours to form another solution.
c. The solution of step (b) is subjected to vigorous stirring, following by drying at temperature of 80° C. for 48 hours to obtain a mixture.
d. The mixture of step (c) is further grinded to obtain very fine powder.
e. The powder of step (d) is dried slowly at temperature of less than 60° C. for 48 hours to obtain a pharmaceutical preparation.

The process further comprises the step of mixing Lanolin and Vaseline in the ratio of 1:1 to the pharmaceutical preparation of step (e) in the concentration of 5% to form an ointment.

Another embodiment of the present invention provides an ointment prepared by mixing Lanolin and Vaseline in the ratio of 1:1 to the pharmaceutical preparation in the concentration of 5%.

In another embodiment of the present invention, the pharmaceutical preparation or ointment have application in the treatment of diabetes and diabetic complications.

Another embodiment of the present invention provides a method of treating diabetes and diabetic complications using Semolina, triticum and chickpea or the pharmaceutical preparation or ointment comprising the same.

Another embodiment of the present invention provides use of the pharmaceutical preparation or ointment as prepared for the treatment of diabetes and diabetic complications.

Yet another embodiment of the present invention provides a process of preparing an ointment from the pharmaceutical preparation as claimed in claim 1, wherein the process comprising the steps of:
a. Mixing flours of Triticum, Chickpea and Semolina with water and stirred vigorously to form a solution.
b. Keeping the solution of step (a) at an appropriate temperature of 27±3° C. for 16 hours to form another solution.
c. The solution of step (b) is subjected to vigorous stirring, following by drying at temperature of 80° C. for 48 hours to obtain a mixture.
d. The mixture of step (c) is further grinded to obtain very fine powder.
e. The powder of step (d) is dried slowly at temperature of less than 60° C. for 48 hours to obtain a pharmaceutical preparation.
f. Mixing the Lanolin and Vaseline in the ratio of 1:1 to the pharmaceutical preparation of step (e) in the concentration of 5% to form an ointment.

In another embodiment of the present invention, the ointment is prepared by mixing Lanolin and Vaseline in the ratio of 1:1 to the pharmaceutical preparation in the concentration of 5%, wherein the ointment is applied 0.5 gm daily over the wounds.

In another embodiment of the present invention, the ointment has application in the treatment of diabetes and diabetic complications, wherein the ointment is applied 0.5 gm daily over the wounds.

Another embodiment of the present invention provides a method of treating diabetes and diabetic complications using Semolina, triticum and chickpea or the pharmaceutical preparation or ointment comprising the same.

Another embodiment of the present invention provides the use of the ointment for the treatment of diabetes and diabetic complications, wherein the ointment is applied 0.5 gm daily over the wounds.

### EXAMPLES

The scope of the present invention is illustrated by the following examples as disclosed herein which are not meant to restrict the scope of the invention in any manner whatsoever.

### EXAMPLE 1: Process of Preparing Pharmaceutical Preparations

The steps comprising of:
a. Mixing flours of Triticum, Chickpea and Semolina with water and stirred vigorously to form a solution.
b. Keeping the solution of step (a) at an appropriate temperature of 27±3° C. for 16 hours to form another solution.
c. The solution of step (b) is subjected to vigorous stirring, following by drying at temperature of 80° C. for 48 hours to obtain a mixture.
d. The mixture of step (c) is further grinded to obtain very fine powder.
e. The powder of step (d) is dried slowly at temperature of less than 60° C. for 48 hours to obtain a pharmaceutical preparation.

### EXAMPLE 2: Preparation of the Ointment

The method comprises of:
a. Mixing flours of Triticum, Chickpea and Semolina with water and stirred vigorously to form a solution.
b. Keeping the solution of step (a) at an appropriate temperature of 27±3° C. for 16 hours to form another solution.
c. The solution of step (b) is subjected to vigorous stirring, following by drying at temperature of 80° C. for 48 hours to obtain a mixture.
d. The mixture of step (c) is further grinded to obtain very fine powder.
e. The powder of step (d) is dried slowly at temperature of less than 60° C. for 48 hours to obtain a pharmaceutical preparation.
f. Mixing the Lanolin and Vaseline in the ratio of 1:1 to the pharmaceutical preparation of step (e) in the concentration of 5% to form an ointment.

### EXAMPLE 3: Experimental Studies

### Preclinical

Pharmaceutical preparation is an Aldose Reductase Inhibitor as well as Alpha Glucosidase inhibitor. It has given 92.39% inhibition in the said enzymes which are responsible for diabetic complications like Retinopathy, Neuropathy and Muscle wreckage.

Studies were conducted to evaluate pharmaceutical preparation in experimental type 1 diabetes (T1DM).

To compare efficacy of pharmaceutical preparation in comparison to standard hypoglycemic agents as insulin. Effects of pharmaceutical preparation on Plasma Glucose.

Pharmaceutical preparation shown very good efficacy in controlling plasma glucose levels.

Studies were conducted to evaluate pharmaceutical preparation in experimental type 2 diabetes (T2DM).

To compare efficacy of this pharmaceutical preparation in comparison to standard hypoglycemic agent pioglitazone.

Effect of pharmaceutical preparation on Plasma Glucose : pharmaceutical preparation has shown comparable efficacy to pioglitazone.

Effect of pharmaceutical preparation on Glycosylated hemoglobin:

Pharmaceutical preparation has shown comparable efficacy to pioglitazone.

TNF alpha effect on Insulin resistance:

To the liver: stimulating the acute phase response, leading to an increase in C-reactive protein and a number of other mediators. It also induces insulin resistance by promoting serine-phosphorylation of insulin receptor substrate-1 (IRS-1), which impairs insulin signaling. So increase in TNF alpha levels means more insulin resistance. Hence, increase in blood glucose levels.

As pharmaceutical preparation is decreasing TNF alpha levels so less insulin resistance and hence regulation of blood glucose levels.

### Toxicity Study Assay Description cl Test System—OECD Acute Oral Toxicity—Up-and-Down-Procedure (UDP)

### Objective: Acute Oral Toxicity (—up-and-down-procedure with LD50)

Test animals: Rats
Strain: Wistar rats

Sex: Female

### General Introduction:

OECD guidelines for the Testing of Chemicals are periodically reviewed in the light of scientific progress or changing assessment practices. The concept of the up-and-down testing approach was first described by Dixon and Mood. In 1985, Bruce proposed to use an up-and-down procedure (UDP) for the determination of acute toxicity of chemicals. There exist several variations of the up-and-down experimental design for estimating an LD50. This guideline is based on the procedure of Bruce as adopted by ASTM in 1987 and revised in 1990. A study comparing the results obtained with the UDP, the conventional LD50 test and the Fixed Dose Procedure (FDP, OECD Test Guideline 420) was published in 1995.

The test consists of a single ordered dose progression in which animals are dosed, one at a time, at a minimum of 48-hour intervals. The first animal receives a dose a step below the level of the best estimate of the LD50. If the animal survives, the dose for the next animal is increased by [a factor of] 3.2 times the original dose; if it dies, the dose for the next animal is decreased by a similar dose progression. Each animal should be observed carefully for up to 48 hours before making a decision on whether and how much to dose the next animal. That decision is based on the 48-hour survival pattern of all the animals up to that time. A combination of stopping criteria is used to keep the number of animals low while adjusting the dosing pattern to reduce the effect of a poor starting value or low slope. Dosing is stopped when one of these criteria is satisfied, at which time an estimate of the LD50 and a confidence interval are calculated for the test based on the status of all the animals at termination.

### Description of the Method

### Selection of Animal Species

Healthy young female rats weighing 110 to 130 grams were selected as the preferred species for conducting the tests in concordance with the paragraph 15 and 16 of OECD TG 425. Healthy young adult nulliparous and nonpregnant female Wistar rats, weighing 110-120 g (10-12 weeks old, at the start of the experiment), were procured.

### Preparation of Animal

The animals were randomly selected and kept in their cages for 7 days prior to dosing to allow for acclimatization to the laboratory conditions. The animals were housed one animal per cage and housed at 22° C. (±3° C.) temperature and relative humidity 45% (±5) with natural day/night cycles (approx 12hr dark/light) and supplied with drinking water, ad libitum and standard rat chow (20 g/animal/day). Clean paddy husk bedding was provided to the animals which was changed every 3^{rd} day.

Main test

Since the LD50 value was not known hence the testing directly proceeded to main test (without conducting the limit tests). The volume given was not more than 2 ml/dose/animal/hr. All animals were fasted overnight prior to dose administration. Following the period of fasting, the fasted BW of each animal was determined, and the dose was calculated according to the BW at the time of dose administration. Food was withheld further for a period of 2-3 hours after completing the dose administration. Single animals were dosed in sequence usually at 48 h intervals and observed daily thereafter, for a total of 14 days. Using the default progression factor, doses were calculated using aot425 software as the assumed LD50. Initial dose (175 mg/Kg BW) was selected based on the recommendations of aot425 software and the subsequent doses were calculated with a dose progression factor of 3.2.

Acute Oral Toxicity (OECD Test Guideline 425) Statistical Program

Test/Substance: Pharmaceutical preparation Test type: Main Test

Limit dose (mg/kg): 2000

Assumed LD50 (mg/kg): Default

Assumed sigma (mg/kg): 0.5

Recommended dose progression: 2000, 550, 175, 55, 17.5, 5.5, 1.75

### Data

TABLE 1

| | Dose | Short-term | Long-term |
|---|---|---|---|
| Seq. ID | (mg/kg) | results | results |
| 1 | 175 | ◯ | ◯ |
| 2 | 550 | ◯ | ◯ |
| 3 | 2000 | ◯ | ◯ |
| 4 | 2000 | ◯ | ◯ |
| 5 | 2000 | ◯ | ◯ |

(X = Died, ◯ = Survived)

Dose Recommendation: The main test is complete.

Stopping criteria met: 3 at Limit Dose.

### Summary of Long-Term Results

TABLE 2

| Dose | ◯ | X | Total |
|---|---|---|---|
| 175 | 1 | 0 | 1 |
| 550 | 1 | 0 | 1 |
| 2000 | 3 | 0 | 3 |
| All | 5 | 0 | 5 |
| doses | | | |

Statistical Estimate based on long term outcomes:

The LD50 is greater than 2000 mg/kg.

### Preparation of Doses

The pharmaceutical preparations were administered as such.

### Doses Used:

As recommended by Acute Oral Toxicity—Up-and-Down-Procedure (UDP), the maximum dose 2000 mg/Kg body weight for testing when there is no estimate of the substance's lethality. The range of selected doses included 175, 500, 2000 mg/Kg of body weight.

### Observations

Body weight

No significant change was observed in individual BW of animals administered the highest allowed dose (2000 mg/kg) for the pharmaceutical preparation.

### Effect of Treatment on the Body Weight of Rats Determined after 14 days of Administering the Highest Dose

TABLE 3

| | | Body | Body | Calculated | |
|---|---|---|---|---|---|
| | Assumed | Weight | weight | t value at | |
| Treatment | LD50(mg/Kg | (Day 0)- | (day 14)- | determined | Remarks |
| group | BW) | in gram | in gram | LD50 | (S or NS) |
| Pharmaceutical | 2000 | 120.000 ± | 130.000 ± | −1.225 | NS* |
| preparation | | 10.000 | 10.000 | | |

S—Significant,
NS—Not significant

### Body Weight of Individual Animals at all Tested Doses

TABLE 4

| | | Body | Body | Body |
|---|---|---|---|---|
| | Dose | Weight | weight | weight |
| Treatment | group | (Day 0) | (day 7) | (day 14) |
| group | (mg/Kg BW) | in grams | in grams | in grams |
| Pharmaceutical | 175 | 110 | 120 | 120 |
| preparation | | | | |
| Pharmaceutical | 550 | 120 | 120 | 130 |
| preparation | | | | |
| Pharmaceutical | 2000 | 130 | 130 | 140 |
| preparation | | | | |
| Pharmaceutical | 2000 | 110 | 110 | 120 |
| preparation | | | | |
| Pharmaceutical | 2000 | 120 | 120 | 130 |
| preparation | | | | |

Note:
All animals survived the test

### Response Tabulation and Toxicity Signs:

TABLE 5

| dose | 30 | 4 | Day | Day | Day | Day | Day | Day | Day | Day | Day | Day | Day | Day | day | day |
|---|---|---|---|---|---|---|---|---|---|---|---|---|---|---|---|---|
| (mg/kg) | min | hr | 1 | 2 | 3 | 4 | 5 | 6 | 7 | 8 | 9 | 10 | 11 | 12 | 13 | 14 |
| 175 | T1 | T1 | T1 | T1 | T1 | T1 | T1 | T1 | T1 | T1 | T1 | T1 | T1 | T1 | T1 | T1 |
| 550 | T1 | T1 | T1 | T1 | T1 | T1 | T1 | T1 | T1 | T1 | T1 | T1 | T1 | T1 | T1 | T1 |
| 2000 | T1 | T1 | T1 | T1 | TS1, | TS1, | TS1, | TS1, | R, | T1 | T1 | T1 | T1 | T1 | T1 | T1 |
| | | | | | T1 | T1 | T1 | T1 | T1 | | | | | | | |
| 2000 | T1 | T1 | T1 | T1 | T1 | T1 | T1 | T1, | R, | T1 | T1 | T1 | T1 | T1 | T1 | T1 |
| | | | | | | | | T1 | T1 | | | | | | | |
| 2000 | T1 | T2, | R, | T1 | T1 | T1 | T1 | T1 | T1 | T1 | T1 | T1 | T1 | T1 | T1 | T1 |
| | | T1 | T1 | | | | | | | | | | | | | |

TOXICITY SIGNS INDEX:
T1—NORMAL
T2—SELFMUTILATION
T2U—SELFMUTILATION LEADING TO ULCERATIVE DERMATITIS
T3—HYPER-ACTIVITY
T4—STRAUB'S TALE
T5—TONIC CLONIC CONVULSIONS
T6—DIMINISHED ACTIVITY
T7—SLEEP
T8—COMA
T9—SKIN AND FUR CHANGES
T10—EYE AND MUCOUS CHANGES
T11—DIARRHOEA
TS1—LOSS OF APPETITE/DECREASEIN FEED CONSUMPTION
M—MORIBUND X-DEATH
R—REVERSAL

### Mortality and LD50

No mortality was observed in any of the animals and at all the tested doses.

### Histopathological Findings

No evident changes for any severe toxicity were observed.

### Results Summary

There was no evidence of any toxicity, at the doses under test. This was confirmed by the histopathological studies. No animal showed mortality or morbidity when the tested dose was administered and even after observation for 14 days.

No toxicity was found in studies which proves that pharmaceutical preparation is safe and can be given at high doses. Based on these findings it may be noted that the provided sample of pharmaceutical preparation did not show any mortality at 2000 mg/Kg body weight

Chemistry, manufacturing, and controls data (CMC)
The raw material is Agro based which is sourced from various parts of the country.
The Final product is obtained through Novelty of Reactions (Biotech).
HPGLC fingerprinting of the raw material ensures consistent quality.
Final product batches monitored by HPGLC.

### Effect of the Pharmaceutical Preparation over Type-1 Diabetes Mellitus

The objective of the study is to evaluate the pharmaceutical preparation in Type-1 Diabetes Mellitus (T1DM) and to compare the efficacy in comparison to standard hypoglycemic agents as insulin.

Experimental model: Streptozotocin (STZ) induced type 1 diabetes (T1DM).

Dose: STZ (45 mg/kg for rats) for T1DM

Route: STZ was administered intraperitoneally (i.p.). Pharmaceutical preparation was administered peroral (p.o.) in three doses.

Volume Administered: Rat—5 ml/kg

Animals: Rats of Wistar or Sprague Dawley strain weighing from 150-200 g of either sex. The animals were housed under optimal laboratory conditions, maintained on a natural (12 h) light and (12 h) dark cycle and have free access to food and water ad libitum. Animals were acclimatized to laboratory conditions before the tests.

Study Design: Animals were divided into 8 groups of 10 animals in each group.
a) Group I: Control or vehicle treated group
b) Group II: Type 1 diabetic group
c) Group III: pharmaceutical preparation (90 mg/kg)+Diabetic animals
d) Group IV: pharmaceutical preparation (180 mg/kg)+Diabetic animals
e) Group V: pharmaceutical preparation (360 mg/kg)+Diabetic animals
f) Group VI: Satcol (60 mg/kg)+Diabetic animals
g) Group VII: pharmaceutical preparation (90 mg/kg)+Insulin (10IU)+Diabetic animals
h) Group VIII: Insulin (10IU)+Diabetic animals

Drugs were administered for 28 days after confirmation of diabetes.

Parameters to be evaluated: The following parameters were evaluated at confirmation of diabetes and after 28 days of treatment.
a) Body weight, water and food intake was monitored weekly.
b) Blood Glucose Level: Glucose was estimated using diagnostic kits by GOD-POD method and the experiment was conducted following the manufacturer's instructions.
c) Oral Glucose Tolerance Test (OGTT): An oral glucose tolerance test was carried out in conscious rats. For this the rats was fasted overnight and glucose (2 g/kg b.w.) was given from 30% solution by oral gavage. Tail blood samples was collected before glucose load and sequentially for every half an hour after glucose load upto 90 minutes and was immediately analysed for glucose.
d) Measurement of Plasma Insulin Level: Insulin was estimated using insulin ELISA kit (LINCO) and the experiment was conducted following the manufacturer's instructions. It is a solid phase sandwich enzyme-linked immunosorbent assay (ELISA), which uses a microtitre plate reader at 450 nm. Concentrations of insulin were calculated from plotted standard curves.

Statistical analysis

All results were expressed as Mean±SEM. The intergroup variation was measured by one-way analysis of variance (ANOVA) followed by Tukey's test. Statistical significance was considered at P<0.05. The statistical analysis was done using the SPSS Statistical Software version 16 (SPSS Inc. 233 South Wacker Drive, 11th Floor Chicago, IL 60606-6412).

### Results

### Effect of Pharmaceutical Preparation on Plasma Glucose (mg/dl)

TABLE 6
Mean Plasma Glucose (mg/dl)

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| Day 1 | 82 ± 2.7 | 334 ± 26.2 | 329 ± 50.8 | 332 ± 50.2 | 327 ± 28.5 | 330 ± 46.0 | 339 ± 33.5 | 352 ± 42.4 |
| Day 7 | 108 ± 17.7 | 355 ± 9.9* | 331 ± 46.4 | 320 ± 38.8 | 269 ± 32.8# | 207 ± 6.1# | 242 ± 13.2# | 249 ± 5.7# |
| Day 14 | 93 ± 4.2 | 397 ± 8.7* | 252 ± 46.6# | 236 ± 35.8# | 219 ± 32.0# | 204 ± 5.4# | 193 ± 10.6# | 222 ± 14.1# |
| Day 21 | 90 ± 6.3 | 396 ± 9.2* | 317 ± 44.7# | 313 ± 35.8# | 262 ± 32.0# | 197 ± 5.8# | 220 ± 9.7# | 233 ± 13.3# |
| Day 28 | 93 ± 4.1 | 465 ± 27.7* | 345 ± 43.4# | 335 ± 31.9# | 305 ± 31.4# | 152 ± 6.4# | 205 ± 9.7# | 211 ± 8.6# |

STZ: Streptozotocin (45 mg/kg; intraperitoneal); Pharmaceutical preparation (90): Pharmaceutical preparation (90 mg/kg; oral gavage); Pharmaceutical preparation (180): Pharmaceutical preparation (180 mg/kg; oral gavage); Pharmaceutical preparation (360): Pharmaceutical preparation (360 mg/kg; oral gavage); Sat (60): Satcol (60 mg/kg; oral gavage); Insulin (10): Insulin (10 IU/kg; subcutaneous).
*p < 0.05 as compared to control;
#p < 0.05 as compared to STZ group.

### Effect of Pharmaceutical Preparation on OGTT—Mean Plasma Glucose (mg/dl)

TABLE 7
OGTT Mean Plasma Glucose (mg/dl)

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| 0.5 hour | 232 ± 28.68 | 772 ± 124.59* | 590 ± 83.67 | 624 ± 93.16 | 494 ± 12.28# | 349 ± 42.21# | 388 ± 59.44# | 389 ± 95.77# |
| 2.0 hour | 166 ± 15.12 | 722 ± 98.89* | 445 ± 69.28# | 378 ± 82.75# | 346 ± 72.33# | 320 ± 38.13# | 282 ± 48.49# | 315 ± 102.13# |
| 4.0 hour | 111 ± 14.75 | 569 ± 120.47* | 442 ± 51.83 | 373 ± 6.73# | 341 ± 4.63# | 227 ± 3.20# | 262 ± 88.15# | 311 ± 217.52 |
| 8.0 hour | 93 ± 9.23 | 395 ± 48.19 | 317 ± 99.96 | 313 ± 79.96 | 262 ± 71.60 | 197 ± 12.96# | 220 ± 21.64# | 233 ± 29.73# |

STZ: Streptozotocin (45 mg/kg; intraperitoneal); Pharmaceutical preparation (90): Pharmaceutical preparation (90 mg/kg; oral gavage); Pharmaceutical preparation (180): Pharmaceutical preparation (180 mg/kg; oral gavage); Pharmaceutical preparation (360): Pharmaceutical preparation (360 mg/kg; oral gavage); Sat (60): Satcol (60 mg/kg; oral gavage); Insulin (10): Insulin (10 IU/kg; subcutaneous).
*p < 0.05 as compared to control;
#p < 0.05 as compared to STZ group.

### Effect of Pharmaceutical Preparation on Body Weight (gm)

TABLE 8
Mean Body Weight (gm)

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| Day 1 | 273 ± 3.0 | 279 ± 4.4 | 284 ± 4.6 | 276 ± 3.6 | 272 ± 5.7 | 278 ± 3.9 | 284 ± 2.5 | 278 ± 3.7 |
| Day 7 | 284 ± 3.2 | 272 ± 4.0 | 283 ± 4.6 | 276 ± 3.6 | 274 ± 5.6 | 278 ± 3.7 | 286 ± 1.9 | 277 ± 4.4 |
| Day 14 | 288 ± 3.0 | 270 ± 4.3* | 281 ± 4.7 | 275 ± 3.7 | 277 ± 5.7 | 280 ± 3.5# | 289 ± 1.8# | 280 ± 3.9 |
| Day 21 | 293 ± 2.7 | 267 ± 4.0* | 280 ± 4.3# | 276 ± 3.4 | 280 ± 5.5# | 282 ± 3.7# | 293 ± 1.7# | 279 ± 4.5# |
| Day 28 | 296 ± 3.3 | 262 ± 3.0* | 281 ± 4.4# | 278 ± 3.5# | 283 ± 6.4# | 284 ± 3.8# | 296 ± 2.0# | 279 ± 4.9# |

STZ: Streptozotocin (45 mg/kg; intraperitoneal); Pharmaceutical preparation (90): Pharmaceutical preparation (90 mg/kg; oral gavage); Pharmaceutical preparation (180): Pharmaceutical preparation (180 mg/kg; oral gavage); Pharmaceutical preparation (360): Pharmaceutical preparation (360 mg/kg; oral gavage); Sat (60): Satcol (60 mg/kg; oral gavage); Insulin (10): Insulin (10 IU/kg; subcutaneous).
*p < 0.05 as compared to control;
#p < 0.05 as compared to STZ group.

### Effect of Pharmaceutical Preparation on Food Intake (gm)

TABLE 9
Food Intake (gm)

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| Day 1 | 17 ± 1.81 | 19 ± 1.81 | 19 ± 0.75 | 20 ± 1.30 | 20 ± 0.58 | 18 ± 1.96 | 20 ± 0.58 | 21 ± 0.66 |
| Day 7 | 19 ± 1.83 | 22 ± 1.59 | 20 ± 0.32 | 21 ± 1.21 | 22 ± 0.71 | 20 ± 1.44 | 22 ± 0.40 | 23 ± 0.43 |
| Day 14 | 20 ± 1.87 | 33 ± 1.81* | 26 ± 1.38# | 25 ± 1.07# | 27 ± 0.58# | 23 ± 1.03# | 23 ± 0.58# | 27 ± 0.58# |
| Day 21 | 21 ± 1.93 | 43 ± 1.05* | 28 ± 1.41# | 32 ± 1.30# | 27 ± 0.58# | 19 ± 1.07# | 23 ± 0.58# | 30 ± 0.56# |
| Day 28 | 19 ± 0.86 | 52 ± 1.59* | 32 ± 1.72# | 37 ± 1.45# | 29 ± 1.21# | 18 ± 0.71# | 26 ± 0.84# | 34 ± 0.93# |

STZ: Streptozotocin (45 mg/kg; intraperitoneal); Pharmaceutical preparation (90): Pharmaceutical preparation (90 mg/kg; oral gavage); Pharmaceutical preparation (180): Pharmaceutical preparation (180 mg/kg; oral gavage); Pharmaceutical preparation (360): Pharmaceutical preparation (360 mg/kg; oral gavage); Sat (60): Satcol (60 mg/kg; oral gavage); Insulin (10): Insulin (10 IU/kg; subcutaneous).
*p < 0.05 as compared to control;
#p < 0.05 as compared to STZ group.

### Effect of Pharmaceutical Preparation on Water Intake (ml)

TABLE 10
Water Intake (ml)

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| Day 1 | 10 ± 0.60 | 12 ± 1.29 | 11 ± 1.07 | 10 ± 0.80 | 10 ± 1.12 | 9 ± 0.86 | 10 ± 0.71 | 9 ± 0.95 |
| Day 7 | 10 ± 0.58 | 22 ± 1.76* | 16 ± 0.66# | 15 ± 0.89# | 12 ± 0.97# | 11 ± 0.89# | 13 ± 0.86# | 14 ± 0.85# |
| Day 14 | 9 ± 0.49 | 31 ± 1.74* | 20 ± 1.03# | 20 ± 1.16# | 17 ± 0.97# | 13 ± 1.85# | 16 ± 1.60# | 20 ± 0.86# |
| Day 21 | 9 ± 0.51 | 50 ± 1.87* | 27 ± 1.30# | 29 ± 2.85# | 22 ± 1.02# | 14 ± 1.44# | 19 ± 2.46# | 25 ± 2.77# |
| Day 28 | 10 ± 1.16 | 63 ± 3.08* | 33 ± 1.86# | 36 ± 3.74# | 30 ± 3.73# | 16 ± 3.11# | 24 ± 2.18# | 31 ± 3.08# |

STZ: Streptozotocin (45 mg/kg; intraperitoneal); Pharmaceutical preparation (90): Pharmaceutical preparation (90 mg/kg; oral gavage); Pharmaceutical preparation (180): Pharmaceutical preparation (180 mg/kg; oral gavage); Pharmaceutical preparation (360): Pharmaceutical preparation (360 mg/kg; oral gavage); Sat(60): Satcol (60 mg/kg; oral gavage); Insulin (10): Insulin (10 IU/kg; subcutaneous).
*p < 0.05 as compared to control;
#p < 0.05 as compared to STZ group.

### Effect of Pharmaceutical Preparation on Urine Output (ml)

TABLE 11
Urine Volume (ml)

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| Day 1 | 7 ± 0.60 | 9 ± 0.49 | 9 ± 0.71 | 10 ± 0.37 | 9 ± 0.68 | 10 ± 0.51 | 10 ± 0.37 | 9 ± 0.63 |
| Day 7 | 8 ± 0.97 | 19 ± 1.92* | 17 ± 1.83 | 18 ± 0.95 | 18 ± 1.20 | 13 ± 1.21# | 15 ± 1.02 | 16 ± 1.91 |
| Day 14 | 9 ± 1.21 | 34 ± 2.86* | 25 ± 2.22 | 28 ± 1.93 | 29 ± 3.09 | 16 ± 2.49# | 21 ± 4.1#0 | 23 ± 2.4#9 |
| Day 21 | 8 ± 0.77 | 45 ± 3.09* | 29 ± 2.87# | 31 ± 3.04# | 30 ± 3.98# | 16 ± 0.93# | 23 ± 3.20# | 26 ± 3.61# |
| Day 28 | 9 ± 0.51 | 59 ± 3.88* | 34 ± 3.65# | 36 ± 3.73# | 32 ± 4.37# | 14 ± 1.44# | 23 ± 3.09# | 26 ± 4.43# |

STZ: Streptozotocin (45 mg/kg; intraperitoneal); Pharmaceutical preparation (90): Pharmaceutical preparation (90 mg/kg; oral gavage); Pharmaceutical preparation (180): Pharmaceutical preparation (180 mg/kg; oral gavage); Pharmaceutical preparation (360): Pharmaceutical preparation (360 mg/kg; oral gavage); Sat (60): Satcol (60 mg/kg; oral gavage); Insulin (10): Insulin (10 IU/kg; subcutaneous).
*p < 0.05 as compared to control;
#p < 0.05 as compared to STZ group.

### Effect of Pharmaceutical Preparation on Plasma Insulin

Percentage Change in plasma insulin

TABLE 12

| | | | | | | | Insulin (10) + | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| Day 1 | 0 | 0 | 0 | 0 | 0 | 0 | 0 | 0 |
| Day 7 | 6 | −80 | −76 | −77 | −80 | −72 | −35 | −42 |
| Day 14 | −1 | −82 | −77 | −79 | −80 | −72 | −41 | −39 |
| Day 21 | 4 | −86 | −79 | −81 | −79 | −72 | −40 | −37 |
| Day 28 | 2 | −87 | −78 | −81 | −77 | −71 | −37 | −34 |

### Findings of the Studies

Streptozotocin led to progressive increase in Blood glucose levels, Food Intake, Water Intake and Urine output. Streptozotocin produced marked reduction in Body Weight.

Effect of pharmaceutical preparation on Plasma Glucose:
Pharmaceutical preparation (90) started showing effect from 7 day onwards
Pharmaceutical preparation (90) shown very good efficacy in controlling plasma glucose levels.
However, Pharmaceutical preparation (90)+Insulin (10) combination showed additive efficacy.
   Effect of pharmaceutical preparation on OGTT:
Oral glucose loading was well-handled by pharmaceutical preparation (90) and pharmaceutical preparation (90)+Insulin (10)
   Effect of pharmaceutical preparation on Body Weight:
Progressive decrease in body weight is correlated with plasma glucose levels.
Pharmaceutical preparation (90) found to be best in controlling body weight in STZ treated rats.
Further, pharmaceutical preparation (90)+Insulin (10) combination showed additional benefit.
   Effect of pharmaceutical preparation on Food Intake:
Progressive increase in food intake is correlated with reduced insulin levels along with decrease glucose uptake from systemic circulation (rise in plasma glucose levels)
Pharmaceutical preparation (90) significantly reduced food intake as compared to other.
Moreover, pharmaceutical preparation (90)+Insulin (10) combination additively reduced food intake.
   Effect of pharmaceutical preparation on Water Intake:
Progressive increase in water intake is correlated with severity of diabetes.
Pharmaceutical preparation (90) restored the water intake in diabetic rats towards normal
   Effect of pharmaceutical preparation on Urine Output:
Progressive increase in urine output is correlated with increased water intake.
Pharmaceutical preparation (90) significantly decreased urine output as compared to other treatment groups
   Effect of pharmaceutical preparation on Plasma Insulin levels:
Marked decline in plasma insulin is correlated with increased plasma glucose levels.
Pharmaceutical preparation did not produce significant changes in plasma insulin levels.

However, plasma glucose levels were significantly reduced at all time points. When we combine pharmaceutical preparation with insulin, pharmaceutical preparation could probably enhance the transportation of insulin to site of action (muscle, liver & adipose tissues) and its consequent binding to insulin receptors so that proper glucose utilization took place. This could be probable reason of reduced systemic insulin levels along with controlled glucose levels. It can be concluded that pharmaceutical preparation is showing insulin sensitizing properties. Thus, pharmaceutical preparation may be demonstrating insulin sensitizing properties like thiazolidinediones (TZDs). TZDs are also shown to reduced plasma insulin levels.

TABLE 13 A
Mean Plasma Glucose (mg/dl)

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| | Control | STZ | (60) | (360) | (180) | (90) | (90) | (10) |
| 6th Week | 96 ± 2.3 | 479 ± 29.0 | 340 ± 48.3 | 334 ± 318 | 314 ± 39.8 | 149 ± 5.9 | 199 ± 10.0 | 226 ± 12.4 |
| 10th Week | 99 ± 2.8 | 485 ± 28.6 | 335 ± 47. | 330 ± 31.5 | 306 ± 35.3 | 145 ± 8.7 | 201 ± 7.9 | 233 ± 16.0 |

TABLE 13 B

| | | | | | | Insulin (10) + | | |
|---|---|---|---|---|---|---|---|---|
| | | | | Pharmaceutical | Pharmaceutical | Pharmaceutical | Pharmaceutical | |
| | | | SAT | preparation | preparation | preparation | preparation | Insulin |
| SEM | Control | STZ | (60) | (360) | (180) | (90) | (90) | (10) |
| 6th Week | 2.3 | 29.0 | 48.3 | 31.8 | 39.8 | 5.9 | 10.0 | 12.4 |
| 10th Week | 2.8 | 28.6 | 47.0 | 31.5 | 35.3 | 8.7 | 7.9 | 16.0 |

### Effect of the Pharmaceutical Preparation Over Type-2 Diabetes Mellitus

The objective of the study is to evaluate pharmaceutical preparation in Type-2 diabetes mellitus (T2DM) and to compare efficacy of these in comparison to standard hypoglycemic agent pioglitazone.

Experimental model: Streptozotocin-nicotinamide induced Type-2 diabetes (T2DM): Single intraperitoneal injection of nicotinamide and streptozotocin was administered.
Dose: STZ (45 mg/kg for rats)+Nicotinamide (230 mg/kg for rats) for T2DM pharmaceutical preparation (45.90 & 180 mg/kg).
Route: STZ & Nicotinamide were administered intraperitoneally (i.p.). Pharmaceutical preparation & pioglitazone were administered per orally (oral gavage).
Volume Administered: Rat—5 mL/kg
Animals: Rats of Wistar strain weighing from 150-200 g of either sex used for the study. The animals were housed under optimal laboratory conditions, maintained on a natural (12 h) light and (12 h) dark cycle and had free access to food and water ad libitum. Animals were acclimatized to laboratory conditions before the tests.
Study Design: Animals were divided into 6 groups of 5-8 animals in each group:
a) Group I: Control or vehicle treated group
b) Group II: Type 2 diabetic group
c) Group III: Diabetic animals+pharmaceutical preparation (45 mg/kg)
d) Group IV: Diabetic animals+pharmaceutical preparation (90 mg/kg)
e) Group V: Diabetic animals +pharmaceutical preparation (180 mg/kg)
f) Group VI: Diabetic animals+Pioglitazone (5 mg/kg)

Pharmaceutical preparation was administered after confirmation of diabetes for 28 days.

Parameters to be evaluated: The following parameters were evaluated after confirmation of diabetes on day 1, 7, 14, 21 and 28 days.
a) Body weight, water and food intake was monitored.
b) Blood Glucose Level: Glucose was estimated using diagnostic kits by GOD-POD method and the experiment was conducted following the manufacturer's instructions.
c) Estimation of Glycosylated Haemoglobin (HbA1c): HbA1c is a good indicator of protein glycosylation. Excess of glucose conjugates with proteins to form glycosylated moieties. It was estimated by ion-resin method using commercial kits.

### Results

### Effect of Pharmaceutical Preparation on Mean Plasma Glucose (mg/dl)

TABLE 14
Mean Plasma Glucose (mg/dl)

| | Control | NAD-STZ | G 45 | G 90 | G180 | Pio |
|---|---|---|---|---|---|---|
| Day 3 | 88 | 140 | 140 | 138 | 141 | 144 |
| Day 7 | 95 | 160 | 178 | 176 | 169 | 149 |
| Day 14 | 95 | 188 | 169 | 152 | 178 | 141 |
| Day 21 | 93 | 190 | 147 | 138 | 175 | 132 |
| Day 28 | 89 | 210 | 137 | 126 | 171 | 126 |

NAD + STZ: Nicotinamide (230 mg/kg; intraperitoneal) + Streptozotocin (45 mg/kg; intraperitoneal); (G45: pharmaceutical preparation (45 mg/kg; oral gavage); G90: pharmaceutical preparation (90 mg/kg; oral gavage); G180: pharmaceutical preparation (180 mg/kg; oral gavage); Pio (5): Pioglitazone (5 mg/kg; oral gavage).
*p < 0.05 as compared to control;
#p < 0.05 as compared to NAD + STZ.

### Effect of Pharmaceutical Preparation on Glycosylated Haemoglobin

TABLE 15
Glycosylated Hemoglobin

| | Control | NAD-STZ | G 45 | G 90 | G180 | Pio |
|---|---|---|---|---|---|---|
| Day 1 | 6.83 | 5.98 | 6.01 | 6.33 | 6.43 | 6.66 |
| Day 7 | 6.88 | 11.22 | 11.34 | 10.89 | 11.42 | 10.78 |
| Day 14 | 6.48 | 12.45 | 11.00 | 9.82 | 10.77 | 9.67 |
| Day 21 | 5.43 | 12.43 | 10.78 | 8.88 | 10.11 | 8.44 |
| Day 28 | 6.68 | 12.89 | 10.24 | 8.45 | 9.89 | 7.96 |

NAD + STZ: Nicotinamide (230 mg/kg; intraperitoneal) + Streptozotocin (45 mg/kg; intraperitoneal); (G45: pharmaceutical preparation (45 mg/kg; oral gavage); G90: pharmaceutical preparation (90 mg/kg; oral gavage); G180: pharmaceutical preparation (180 mg/kg; oral gavage); Pio (5): Pioglitazone (5 mg/kg; oral gavage).
*p < 0.05 as compared to control;
#p < 0.05 as compared to NAD + STZ.

### Findings of the Study:

a) Nicotinamide-Streptozotocin led to progressive increase in Blood glucose levels and Glycosylated hemoglobin.

b) Effect of pharmaceutical preparation on Plasma Glucose:
Pharmaceutical preparation reduced plasma glucose levels & the effect was evident from 14th day onwards.
Pharmaceutical preparation (90) has shown comparable efficacy to pioglitazone (5).

c) Effect of pharmaceutical preparation on Glycosylated hemoglobin:
Progressive increase in blood glucose is correlated with increased glycosylated hemoglobin.
Pharmaceutical preparation reduced glycosylation of hemoglobin and they started showing effect from 14th day onwards.
Pharmaceutical preparation (90) has shown comparable efficacy to pioglitazone (5).

### Studies on Ointment

### Materials and Methods

Experimental animals

Adult female wistar rats weighing 250-300 g were used. All animals were maintained on a 12 h light/dark cycle under standard conditions (25±2° C., 60-70% humidity). They were fed with a commercial standard rat diet and water ad libitum. 7 days prior to the start of the experiment, the ratswere acclimatized to the animal house environment. The experimental protocol was approved by Institutional Animal Ethics Committee (IAEC). All experiments were performed according to the guidelines of Committee for the Purpose of Control and Supervision of Experimentation on Animals (CPCSEA), Government of India.

Preparation of the ointment

Lanolin and Vaseline were mixed in the ratio of 1:1 and pharmaceutical preparation was added in concentration of 5% to prepare the ointment. The wounds of rats were treated with 0.5 g of the ointment daily during the course of treatment

Chemicals and reagents

Pharmaceutical preparation, Cipladine (Povidone iodine 5%) was purchased from CIPLA India. Ltd, Enzyme Linked Immunosorbent Assay (ELISA) kits for TNF-α and IL-6 were obtained from R & D Systems, Minneapolis MN, USA respectively. NF-κβ, NRF-2 and HO-1 sandwich ELISA kit was procured from Elab science, China.

Induction of diabetes

Single intraperitoneal injection of streptozotocin (STZ) freshly prepared in citrate buffer (0.1M, pH4.5), at a concentration of 55 mg/kg body weight, was used to induce experimental hyperglycemia in overnight-fasted rats. Control rats were administered citrate buffer instead. To counter hypoglycaemic shock rats were given glucose (5% w/v) solution during the first 24 h. Blood glucose levels were measured seventy-two hours later, using FreeStyl Optimum Neo® (Abbott Diabetes Care Ltd., UK) digital glucometer from the tail puncture on the tip.

Animals with blood glucose level≥250 mg dL-1 were defined as hyperglycemic and kept under observation or another 7 days. Then, the hyperglycemic rats that maintained high glycemic levels were considered for the in vivo experiments.

### Results

### Effect on Percentage of Wound Closure

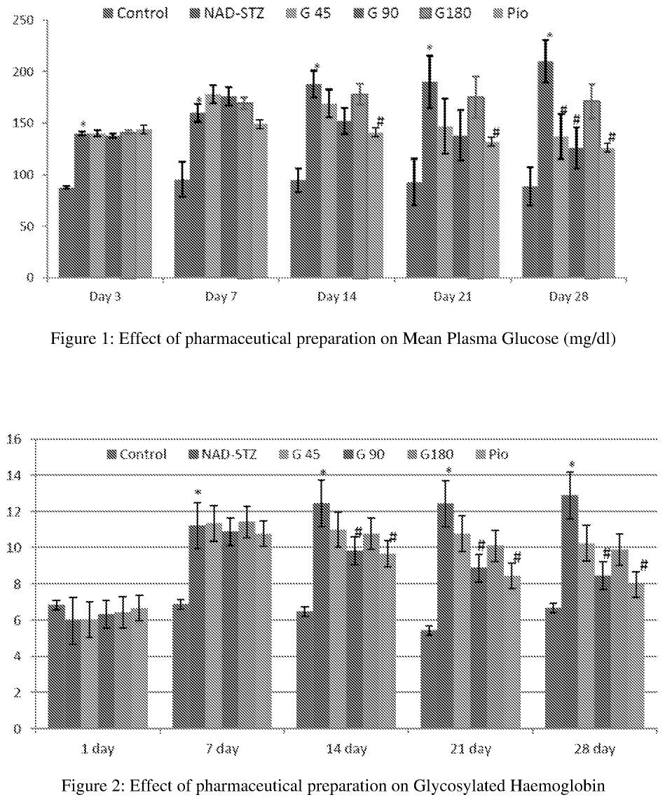

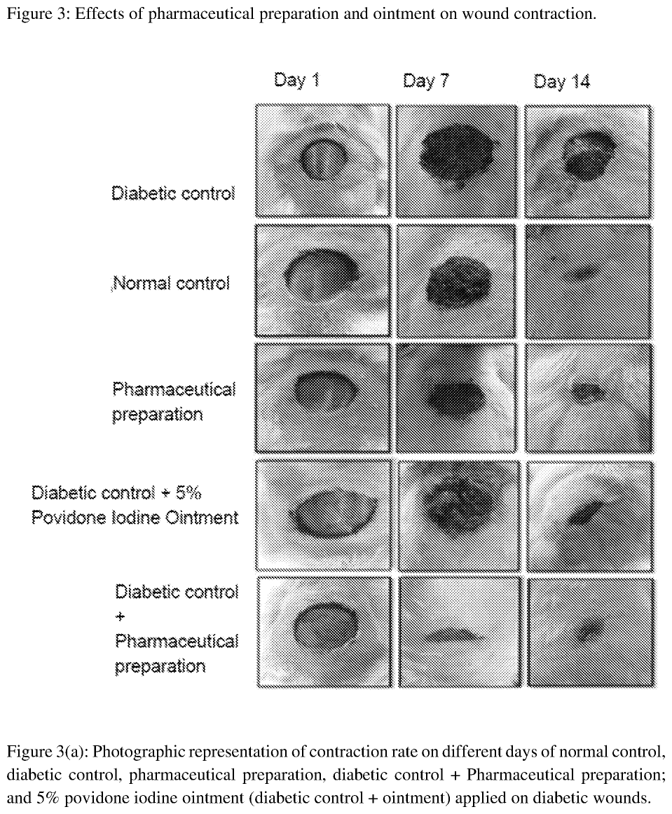

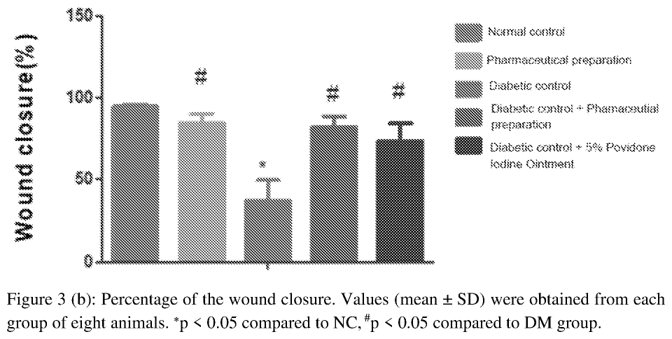

FIG. 3 depicts the variations in wound closure at first, seventh and fourteenth day in control and experimental groups. The wound closure was significantly delayed on 7th and 14th days in diabetic control rats when compared to non-diabetic rats. The % wound closure in diabetic control rats was significantly improved on day 7th and 14th in pharmaceutical preparation and ointment treated rats

### Conclusion

The present study was designed to explore the wound healing potential of pharmaceutical preparation in STZ induced diabetic rats.
Pharmaceutical preparation administration significantly attenuated oxidative stress induced by hyperglycaemia and showed strong antioxidant activity by increasing the expression of Nrf2 and HO-1 through which the levels of antioxidant enzymes like GSH, GPx, SOD, catalase was also increased.
The elevated level of inflammatory markers like TNF-60 , NFκB, IL-1β, IL-6 were also decreased by pharmaceutical preparation administration, this showed the anti-inflammatory activity of pharmaceutical preparation.
Collagen synthesis was also increased by pharmaceutical preparation that was assessed by hydroxyproline assay.
In the conclusion, topical application of the prepared ointment showed wound healing effect in diabetic rats by its anti-inflammatory, antioxidant, and anti-diabetic activities.

### Certificate of Analysis

### Clinical Pharmacology:

Pharmaceutical preparation is administered orally. It is well absorbed. As observed from Human data, it does not contradict nor create negativity with Food and other drugs. All blood parameters remain normal and patients who were having medications for Thyroid, Heart, neuro-defects, rheumatic diseases, Painkillers like (Ultracet, Tramadol), liver diseases were continuing taking this preparation along with their prescribed medication. Usually, no adverse effects were reported. The pharmacokinetics is not altered in patients with renal or liver diseases. The above all information is based on clinical study performed in patients.

1. A process of preparing a pharmaceutical preparation, wherein the process comprising the steps of:
a) Mixing flours of Triticum, Chickpea and Semolina with water and stirred vigorously to form a solution;
b) Keeping the solution of step (a) at an appropriate temperature range of 25° C. to 35° C. for an adequate time ranging 12 to 19 hours to form another solution;
c) Subjecting the solution of step (b) to stirring, followed by drying at temperature range of 70° C. to 90° C. for a time range of 40 to 60 hours to obtain a mixture;
d) Grinding the mixture of step (c) to obtain a powder; and
e) Drying the powder of step (d) at temperature range of 0° C. to 65° C. for a time range of 42 to 55 hours to obtain the pharmaceutical preparation.

2. The process as claimed in claim 1, wherein flours of Triticum, Chickpea and Semolina are obtained by grinding grains of Triticum, Chickpea and Semolina, respectively.

3. The process as claimed in claim 1, wherein the water is distilled water.

4. (canceled)

5. The process as claimed in claim 1, wherein Triticum, Semolina and chickpea flour and water are mixed in a ratio of 1:1 to 1.

6. The process as claimed in claim 1, wherein the appropriate temperature range of step (b) is 26° C. to 32° C.

7. (canceled)

8. The process as claimed in claim 1, wherein the adequate time of step (b) ranges from 15 to 18 hours.

9. (canceled)

10. The process as claimed in claim 1, where drying of step (c) is carried out at temperature range of 75 to 85° C.

11. The process as claimed in claim 10, wherein the temperature of step (c) is 80° C.

12. The process as claimed in claim 1, wherein the time range of step (c) is 45 to 55 hours.

13. The process as claimed in claim 1, wherein the temperature of step (e) is less than 62 ° C.

14. The process as claimed in claim 1, wherein the time range of step (e) is 46 to 52 hours.

15. The process as claimed in claim 1, wherein the pharmaceutical preparation has application in treatment of diabetes and diabetic complications.

16. (canceled)

17. Use of the pharmaceutical preparation as prepared in process of claim 1 for the treatment of diabetes and diabetic complications.

18. A process of preparing a pharmaceutical preparation, wherein the process comprising the steps of:
a. Mixing flours of Triticum, Chickpea and Semolina with water and stirring to form a solution.
b. Keeping the solution of step (a) at an appropriate temperature of 27'3° C. for 16 hours to form another solution.
c. Subjecting the solution of step (b) to stirring, followed by drying at temperature of 80° C. for 48 hours to obtain a mixture.
d. Grinding the mixture of step (c) to obtain powder.
e. Drying the powder of step (d) at temperature of less than 60° C. for 48 hours to obtain a pharmaceutical preparation.

19. (canceled)

20. (canceled)

21. (canceled)

22. A process of preparing an ointment from the pharmaceutical preparation as claimed in claim 1, wherein the process comprising the steps of:
a. Mixing flours of Triticum, Chickpea and Semolina with water and stirring to form a solution;
b. Keeping the solution of step (a) at an appropriate temperature of 27+3° C. for 16 hours to form another solution;
c. Subjecting the solution of step (b) to stirring, followed by drying at temperature of 80° C. for 48 hours to obtain a mixture;
d. Grinding the mixture of step (c) to obtain powder;
e. Drying the powder of step (d) at temperature of less than 60° C. for 48 hours to obtain the pharmaceutical preparation; and
f. Mixing Lanolin and petroleum jelly in the ratio of 1:1 to the pharmaceutical preparation of step (e) in the concentration of 5% to form the ointment.

23. The ointment of claim 1, wherein the ointment is applied 0.5 gm daily over the wounds.

24. (canceled)

25. (canceled)

26. (canceled)
